(19) United States
(12) Patent Application Publication
(10) Pub. No.: US 2014/0228818 A1
(43) Pub. Date: Aug. 14, 2014

(54) URETHRAL CATHETER TENSION HOLDING AND MOVEMENT STABILIZING DEVICES

(71) Applicant: Thomas S. Brinton, Sandy, UT (US)

(72) Inventor: Thomas S. Brinton, Sandy, UT (US)

(73) Assignee: MAYO FOUNDATION FOR MEDICAL EDUCATION AND RESEARCH, Rochester, MN (US)

(21) Appl. No.: 14/239,050

(22) PCT Filed: Aug. 15, 2012
(86) PCT No.: PCT/US2012/050857

§ 371 Date: Apr. 23, 2014

Related U.S. Application Data
(60) Provisional application No. 61/524,075, filed on Aug. 16, 2011.

(51) Int. Cl. A61M 25/00; A61M 25/02

(52) U.S. Cl. CPC A61M 25/0017; A61M 25/02; A61M 2025/0213; A61M 2210/1483; A61M 2210/167; A61M 2202/0014; A61M 2202/0496
USPC 604/544

(57) ABSTRACT

This document provides methods and materials involved in holding tension on a urethral catheter and/or stabilizing the movement of a urethral catheter. For example, devices configured to allow a physician to hold tension on a urethral catheter and stabilize movement of a urethral catheter at the urethral meatus in a mammal (e.g., a human) in a non-invasive manner as well as methods for using catheter tension holding and movement stabilizing devices are provided.

### CROSS REFERENCE TO RELATED APPLICATIONS

This application claims the benefit of U.S. Provisional Application Ser. No. 61/524,075, filed Aug. 16, 2012. The disclosure of the prior application is considered part of (and is incorporated by reference in) the disclosure of this application.

### BACKGROUND

1. Technical Field

This document relates to methods and materials involved in holding tension on a urethral catheter and/or stabilizing the movement of a urethral catheter. For example, this document relates to devices configured to allow a physician to hold tension on a urethral catheter and to stabilize movement of a urethral catheter at the urethral meatus in a mammal (e.g., a human) in a non-invasive manner.

2. Background Information

Catheters are medical devices that can be used to facilitate various medical procedures. A catheter can be inserted into the body of a patient and thereby allow drainage or removal of fluids. For example, a typical urethral catheter is a Foley catheter. A Foley catheter is a flexible tube that is passed through the urethra and into the bladder. The catheter tube can have two lumens running along its length. One lumen is open at both ends allowing urine to drain out into a collection bag. The other lumen has a valve on the outside end and connects to a retention balloon at the tip. The retention balloon is inflated when it lies inside the bladder, to stop it from slipping out. The catheter tubing can be gently pulled on to apply force on the retention balloon.

### SUMMARY

This document provides methods and materials involved in holding tension on a urethral catheter and/or stabilizing the movement of a urethral catheter. For example, this document provides devices configured to allow a physician to hold tension on a urethral catheter and stabilize movement of a urethral catheter at the urethral meatus in a mammal (e.g., a human) in a non-invasive manner as well as methods for using catheter tension holding and movement stabilizing devices.

In some cases, it can be difficult to maintain tension on a urethral catheter. A catheter is often taped or strapped to a patient's leg in an attempt to maintain tension on the catheter. This can be uncomfortable for a patient, and the tape and catheter may not remain in place whereby the desired tension is lost. As described herein, catheter tension holding and movement stabilizing devices can be designed such that they attach to the urethral catheter tubing external to the urethral meatus. Urethral catheter tension holding and movement stabilizing devices can hold the catheter tubing tightly, allow tension to be applied to the catheter, and prevent the catheter tubing from sliding, thereby holding the desired tension on the catheter. Having the ability to prevent the catheter tubing from sliding in and out of the urethral meatus can also allow the physician to non-pharmacologically decrease the pain and discomfort associated with this sliding in and out motion for the patient. Reducing urethral catheter discomfort and pain to the patient when they move can give the patient more encouragement to move about and become ambulatory post-surgery. Having the ability to hold tension on a urethral catheter can allow the physician to treat medical conditions that respond to compression of the bladder neck and the organs that lie directly beneath the bladder (e.g., the male prostate) due to force applied to the retention balloon at the distal end of the urethral catheter. For example, hematuria (blood in the urine) of suspected prostatic origin, leaky anastomosis after prostatectomy, urine leakage around the catheter with bladder spasm and instilling intravesical chemotherapy after transurethral resection of bladder tumor (TURBT) are medical conditions that all respond positively to tension held on a urethral catheter. Having the ability to minimize catheter tube movement and/or apply and hold tension on a urethral catheter as described herein can relieve discomfort of catheterized patients and help aid in recovery of catheterized patients.

In general, one aspect of this document features a device for holding tension on and stabilizing the movement of a urethral catheter. The device comprises, or consists essentially of, two separable half components configured to be attached to a urethral catheter tubing, thereby forming the device, wherein the device is configured to contain or rest against the penis glans or rest against the labia minora, labia majora or other perineal structure, wherein at least one of the two separable half components comprises a fastener configured to hold the two separable half components together, and wherein formation of the device forms a continuous open tube configured to retain at least a portion of the urethral catheter tubing. An inner surface of the continuous open tube configured to retain at least a portion of the urethral catheter tubing can be coated. An inner surface of the continuous open tube configured to retain at least a portion of the urethral catheter tubing can be coated with a material selected from the group consisting of polyvinyl chloride (PVC), silicone, rubber, latex, Teflon, nylon, adhesives, and abrasives. An inner surface of the continuous open tube configured to retain at least a portion of the urethral catheter tubing can be not smooth. An inner surface of the continuous open tube configured to retain at least a portion of the urethral catheter tubing can comprise a ring, a bump, teeth, a hook, or a pin. The fastener can comprise a complimentary male protrusion and female indentation. The fastener can comprise at least two magnets. The device can comprise material selected from the group consisting of polyvinyl chloride (PVC), silicone, rubber, latex, Teflon, and Nylon. A diameter of the continuous open tube configured to retain at least a portion of the urethral catheter tubing can be between 8 French and 30 French. The diameter can be between 18 French and 24 French.

In another aspect, this document features a kit comprising a device for holding tension on and stabilizing the movement of a urethral catheter as described in the preceding paragraph and a urethral catheter. The urethral catheter comprises a retention balloon near a tip of the catheter.

In another aspect, this document features a device for holding tension on and stabilizing the movement of a urethral catheter. The device comprises, or consists essentially of, two separable half components configured to be attached to a urethral catheter tubing, thereby forming the device, wherein the device is configured to sit external to the urethral meatus of a human patient, wherein formation of the device forms a lumen, wherein the lumen is configured to retain at least a portion of the urethral catheter tubing, wherein the two separable half components are configured to contain at least one hook configured to grip the catheter tubing, wherein force applied to the external surface of the device moves the hook so as the hook releases the catheter tubing, wherein the device displays at least two markings on the external surface, and wherein the markings indicate where the force is to be applied. An inner surface of the lumen can be coated. An inner surface of the lumen can be coated with a material selected from the group consisting of polyvinyl chloride (PVC), silicone, rubber, latex, Teflon, Nylon, adhesives, and abrasives. The hook can comprise metal wire or plastic. The device can comprise material selected from the group consisting of polyvinyl chloride (PVC), silicone, rubber, latex, Teflon, and Nylon. A diameter of the lumen can be between 8 French and 30 French. A diameter of the lumen can be between 18 French and 24 French.

In another aspect, this document features a kit comprising a device for holding tension on and/or stabilizing the movement of a urethral catheter as described in the preceding paragraph and a urethral catheter. The urethral catheter comprises a retention balloon near a tip of the catheter.

In another aspect, this document features a device for holding tension on and stabilizing the movement of a urethral catheter. The device comprises, or consists essentially of, a flexible proximal component and a rigid distal component configured to be attached to a urethral catheter tubing, thereby forming the device, wherein the device is configured to sit external to the urethral meatus of a human patient, wherein the distal component is configured to retain at least a portion of the urethral catheter tubing, and wherein the proximal component separates the distal component and the urethral meatus of the patient. The proximal component can comprise material selected from the group consisting of polyvinyl chloride (PVC), silicone, rubber, latex, Teflon, and Nylon. The distal component can comprise material selected from the group consisting of acrylic (plexiglass), polycarbonate, polyvinyl, flouroelastomers, acrylates, polystyrene, polyaryletheretherketone, polyphenylene sulphide, polypropylene, polyvinyl chloride (PVC), silicone, rubber, latex, Teflon, and Nylon.

In another aspect, this document features a kit comprising a device for holding tension on and/or stabilizing the movement of a urethral catheter as described in the preceding paragraph and a urethral catheter. The urethral catheter comprises a retention balloon near a tip of the catheter.

In another aspect, this document features a device for holding tension on a urethral catheter. The device comprises, or consists essentially of, a single component, wherein the component is configured to be attached to a urethral catheter tubing, to retain at least a portion of the urethral catheter tubing, and to sit external to the urethral meatus of a human patient. The component can comprise a lumen configured to retain at least a portion of the urethral catheter tubing, wherein not all sides of the lumen contact the urethral catheter tubing, wherein a cross sectional length of the lumen is greater than a cross sectional width of the lumen, wherein the lumen is configured to have an open state so as no sides of the lumen contact the urethral catheter tubing when force is applied to an external surface of the component, wherein the component displays at least two markings on the external surface to indicate where the force is to be applied. The component can comprise an inflated balloon, wherein the inflated balloon is configured to wrap around the urethral catheter tubing so as to attach to itself. The component can comprise a clip. An inner surface of the clip can be coated. An inner surface of the clip can be coated with a material selected from the group consisting of polyvinyl chloride (PVC), silicone, rubber, latex, Teflon, Nylon, adhesives, and abrasives.

In another aspect, this document features a kit comprising a device for holding tension on and/or stabilizing the movement of a urethral catheter as described in the preceding paragraph and a urethral catheter. The urethral catheter comprises a retention balloon near a tip of the catheter.

In another aspect, this document features a method of holding tension on and stabilizing the movement of a urethral catheter. The method comprises, or consists essentially of, applying tension on the urethral catheter and applying a urethral catheter tension holding and/or movement stabilizing device described herein external to the urethral meatus of a patient.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a side-view diagram of the male urinary tract with a Foley catheter in place together with a urethral catheter tension holding and movement stabilizing device in accordance with some embodiments.

FIG. 1B is a side-view diagram of the female urinary tract with a Foley catheter in place together with a urethral catheter tension holding and movement stabilizing device in accordance with some embodiments.

FIG. 2 is a mid cross sectional view of an exemplary urethral catheter tension holding and movement stabilizing device in accordance with some embodiments.

FIG. 3A is a top view of the device of FIG. 2.

FIG. 3B is a bottom view of the device of FIG. 2.

FIG. 4A is a 180 degree profile view of the device of FIG. 2.

FIG. 4B is a top view of one half of the device of FIG. 2.

FIG. 5A is a top view of an exemplary urethral catheter tension holding and movement stabilizing device in accordance with some embodiments.

FIG. 5B is a top view of one half of an exemplary urethral catheter tension holding and movement stabilizing device in accordance with some embodiments.

FIG. 6A is a top view of the proximal component of an exemplary urethral catheter tension holding and movement stabilizing device in accordance with some embodiments.

FIG. 6B is a perspective view of the distal component of an exemplary urethral catheter tension holding and movement stabilizing device in accordance with some embodiments.

FIG. 7A is a top view of an exemplary urethral catheter tension holding and movement stabilizing device in accordance with some embodiments.

FIG. 7B is a top view of the device of FIG. 7A with applied force.

Like reference symbols in the various drawings indicate like elements.

### DETAILED DESCRIPTION

This document provides methods and materials involved in holding tension on a urethral catheter. For example, this document provides devices configured to allow a physician to hold tension on a urethral catheter in a mammal (e.g., a human) in a non-invasive manner as well as methods for using catheter tension devices.

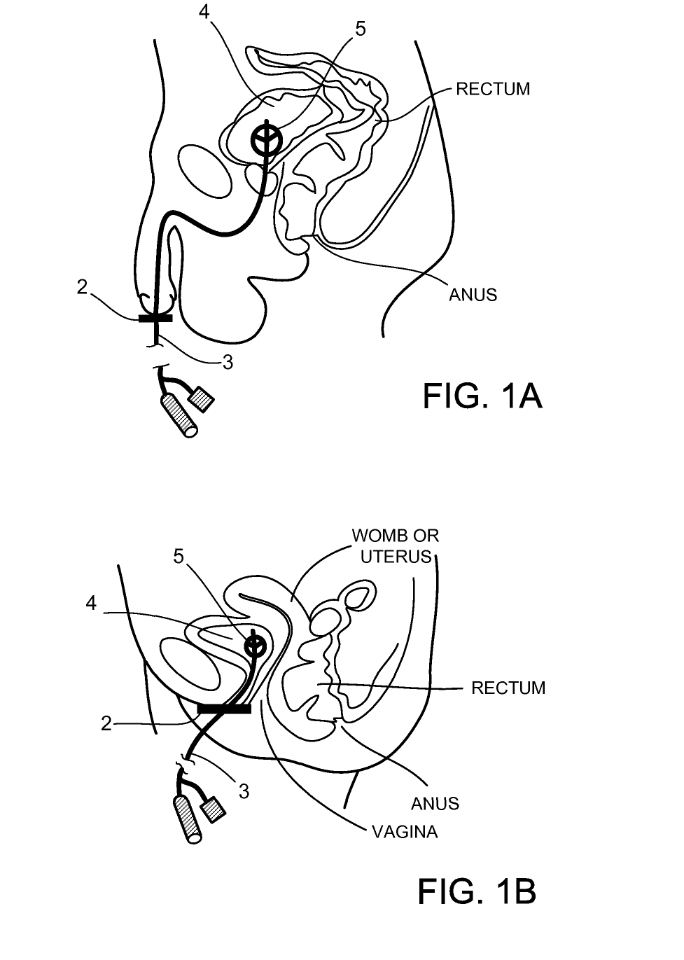

In general, a urethral catheter tension and movement stabilizing device can be configured to have a tight hold on the urethral catheter tubing. A urethral catheter tension and movement stabilizing device can be placed on the catheter tubing when a physician pulls on a urethral catheter to apply tension on the retention balloon located in the patient's urinary bladder. With reference to FIG. 1, placement of a urethral catheter tension holding and movement stabilizing device 2 external to the urethral meatus can prevent catheter tubing 3 from sliding inward towards bladder 4. The urethral catheter tension holding and movement stabilizing device 2 can create a fixed point external to the urethral meatus while a urethral catheter retention balloon 5 located within bladder 4 creates a second fixed point. The catheter tubing between the two fixed points can be held in tension.

In some cases, a urethral catheter tension holding and movement stabilizing device can have a lumen that the urethral catheter tubing passes through. The diameter of the lumen can be configured to fit different size urethral catheters. For example, the lumen can be between about 8 French and 30 French. In some cases, the surface of the lumen walls can be made irregular to increase resistance to sliding of the catheter tubing with configurations such as, without limitation, constriction rings, bumps, hooks, points, teeth, or pins. In some cases, the surface of the lumen walls of a urethral catheter tension holding and movement stabilizing device can be abrasive such that there is an increase in resistance to sliding of the catheter tubing. In some cases, the surface of the lumen walls can be lined with a material to increase resistance to sliding of the catheter tubing such as, without limitation, a thin layer of silicone rubber or adhesive. In some cases, the surface of the lumen walls can have magnetic strips, magnetic points, or other magnetic configurations to increase resistance to sliding of the catheter tubing within the lumen of a urethral catheter tension holding and movement stabilizing device. In some cases, the surface of the lumen walls can have rings or bands to decrease the diameter of the lumen thereby providing increased resistance to sliding of the catheter tubing.

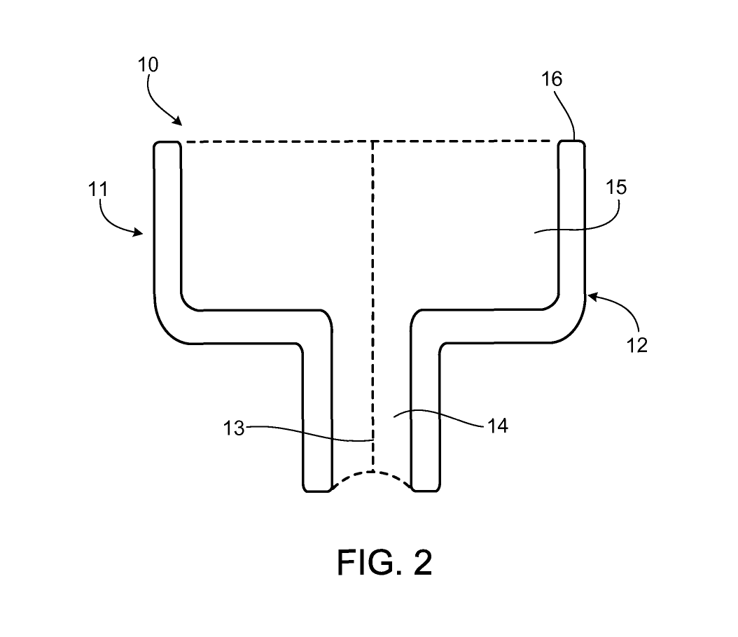

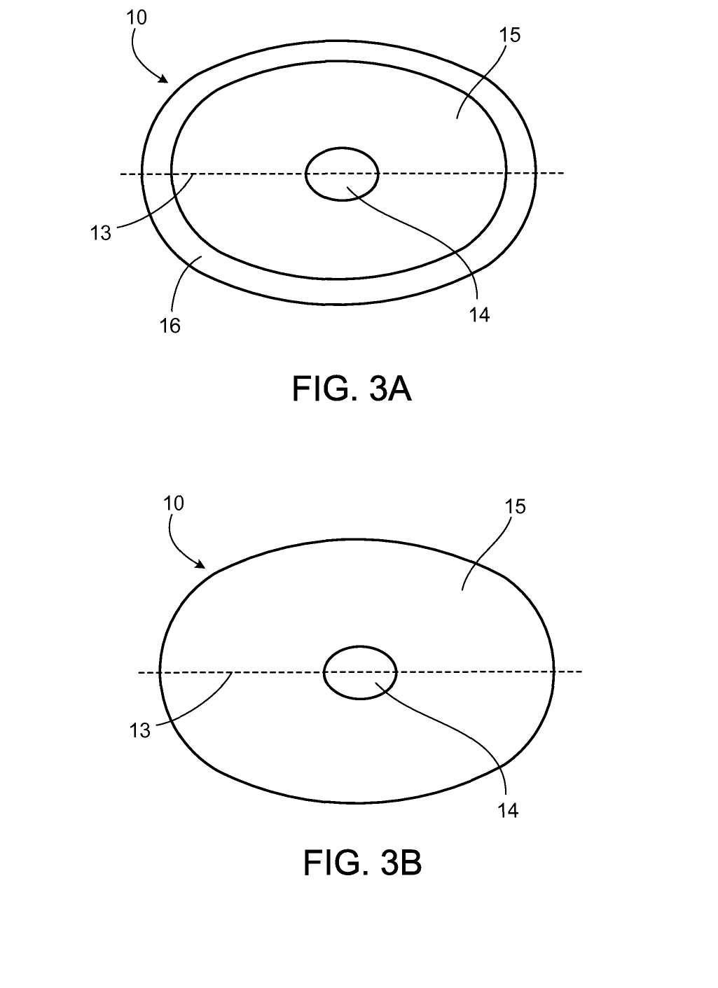

As described herein, a urethral catheter tension holding and movement stabilizing device can be composed of two separable portions or halves that connect to one another. With reference to FIGS. 2, 3, and 4, a device 10 can include two portions (e.g., halves) 11 and 12. Line 13 can represent the junction between two portions 11 and 12. Device 10 can have a proximal end that lies next to the urethral meatus during use and a distal end. When two portions 11 and 12 are connected, they can form a lumen 14 at the distal end. A urethral catheter can pass through lumen 14. The diameter of lumen 14 can vary to hold different size urethral catheters firmly. A device 10 comprising two connected portions 11 and 12 can have a receptacle 15 at or near the proximal end. Receptacle 15 can be configured contain the penis glans. In some cases, a receptacle rim 16 can be configured to rest against a labia minora, a labia majora, or other perineal structure during use. The diameter of receptacle 15 can range from about 1 cm to about 10 cm (e.g., from about 1 cm to about 8 cm, from about 1 cm to about 6 cm, from about 2 cm to about 10 cm, from about 4 cm to about 10 cm, from about 5 cm to about 10 cm, or from about 6 cm to about 10 cm). The wall thickness of receptacle 15 and lumen 14 can range from about 1 mm to about 50 mm (e.g., from about 5 mm to about 50 mm, from about 10 mm to about 50 mm, from about 15 mm to about 50 mm, from about 1 mm to about 45 mm, from about 1 mm to about 40 mm, from about 1 mm to about 35 mm, or from about 10 mm to about 40 mm). The width of receptacle rim 16 can range from about 1 mm to about 50 mm (e.g., from about 5 mm to about 50 mm, from about 10 mm to about 50 mm, from about 15 mm to about 50 mm, from about 1 mm to about 45 mm, from about 1 mm to about 40 mm, from about 1 mm to about 35 mm, or from about 10 mm to about 40 mm). In some cases, rim 16 can be rounded at the corners for patient comfort. Receptacle 15 of device 10 can be any suitable shape so as to position device 10 external to the urethral meatus. For example, the proximal end of device 10 can be a shape such as, without limitation, a disc, a semi-circular cup, a polygonal shape with or without rounded corners, or any other appropriate shape as to contain the penis glans, rest against the labia minora, labia majora, or any other perineal structure, or rest against the urethral meatus. Receptacle 15 of device 10 can have perforations in its wall. Receptacle 15 of device 10 can have a lining on the inner surface of its wall. The open proximal end of device 10 and the open distal end of device 10 can create one continuous passage. The length of lumen 14 can range from about 1 mm to about 100 mm (e.g., from about 10 mm to about 100 mm, from about 20 mm to about 100 mm, from about 30 mm to about 100 mm, from about 1 mm to about 90 mm, from about 1 mm to about 80 mm, from about 1 mm to about 70 mm, from about 25 mm to about 75 mm, or from about 10 mm to about 50 mm) The length of receptacle 15 can range from flat to about 10 cm (e.g., from flat to about 9 cm, from flat to about 8 cm, from flat to about 7 cm, or from flat to about 6 cm).

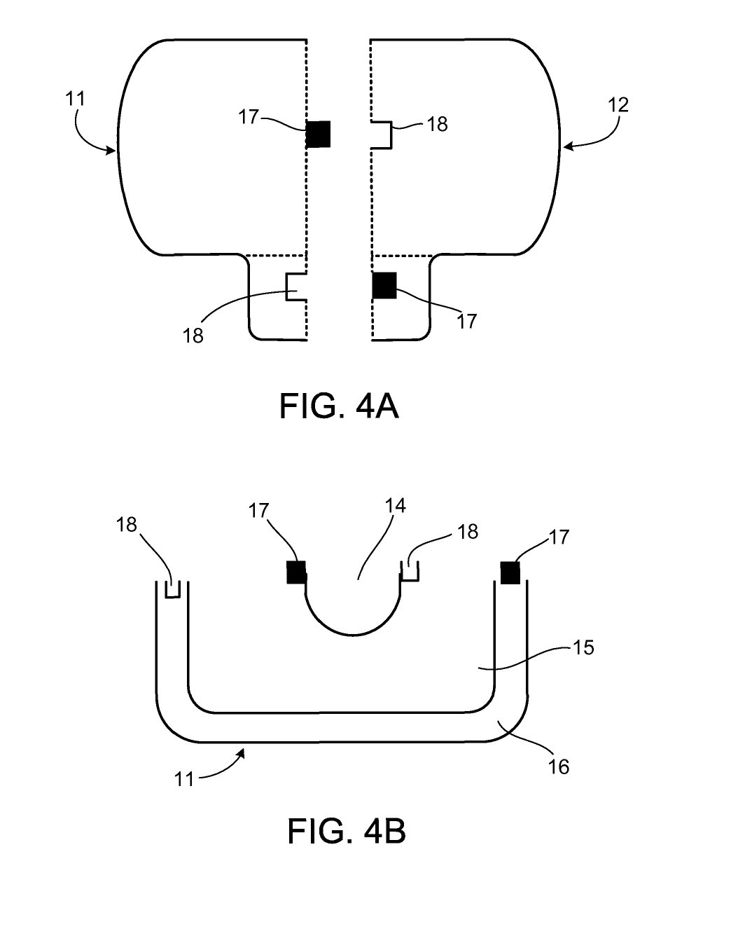

Two portions 11 and 12 of urethral catheter tension device 10 can connect by any appropriate means. For example, two portions 11 and 12 can contain one or more complimentary male fasteners 17 and female fasteners 18 as shown in FIG. 4. Male fasteners 17 can be any suitable shape such that they can be received by female fasteners 18. In some cases, portion 11 and portion 12 can be connected together by magnets incorporated into wall edges of portion 11 and portion 12 where they connect together.

A device provided herein to hold tension on a urethral catheter can be composed of any appropriate material. For example, device 10 can be composed of material such that it has a rigid or semi-rigid structure. Examples of materials that can be used to make a device provide herein (e.g., device 10 or device 40) include, without limitation, polyvinyl chloride (PVC), silicone rubber, latex, Teflon, nylon, and other appropriate material.

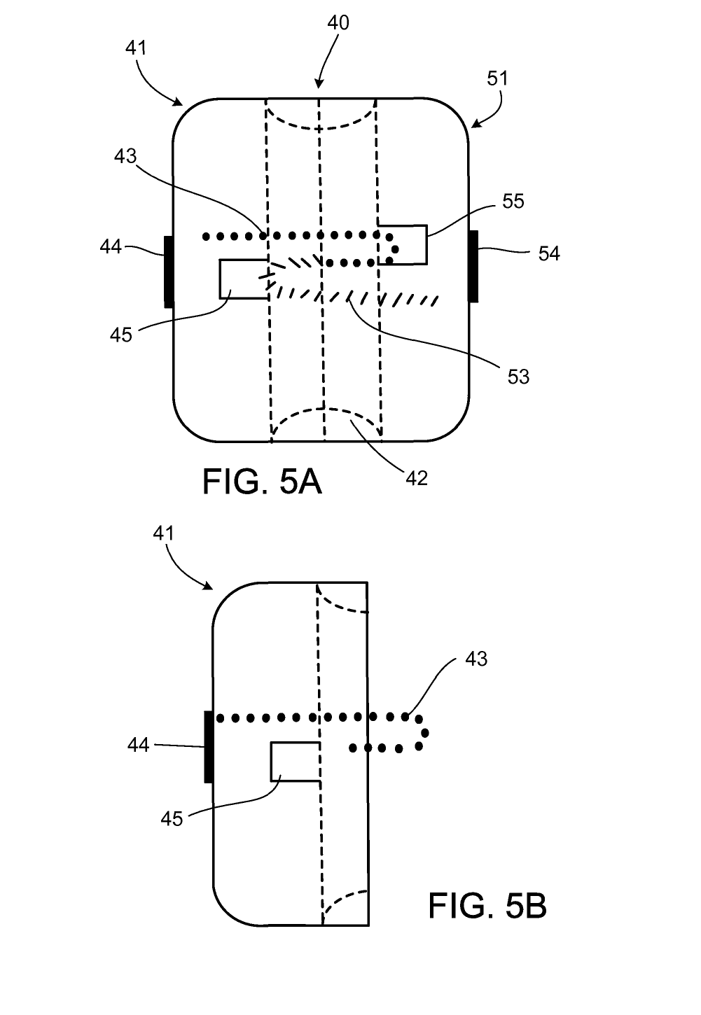

In some cases, a urethral catheter tension holding and movement stabilizing device can be composed of two individual portions (e.g., halves) that can grip catheter tubing. For example, as shown in FIG. 5, device 40 can be composed of two portions 41 and 51. When portions 41 and 51 are adjacent to one another, they can form a lumen 42 that runs longitudinally between them configured to contain at least a portion of a urethral catheter tubing. Portions 41 and 51 can each contain one or more hooks (e.g., a hook 43 of portion 41, and a hook 53 of portion 51) that extend from within the walls of portion 41 or 51 such that the hooks (e.g., hooks 43 and 53) wrap around a catheter tubing, thereby holding it securely in position. Hooks 43 and 53 can be of any appropriate shape and size so as to fit firmly around a urethral catheter. Portions 41 and 51 can have markings 44 and 54 located on an outer wall to indicate where the end of hook 43 and hook 53 are located within the walls of portions 41 and 51. Force can be applied to markings 44 and 54 to cause hooks 43 and 53 to move forward away from the catheter tubing, thus releasing the grip on the tubing. Portions 41 and 51 can contain hollow pockets 45 and 55 of the appropriate size and shape to provide room for hooks 43 and 53 when they move forward, releasing their hold on the catheter tubing. A pocket on one portion can be configured to receive a hook from the other portion. For example, pocket 45 can be configured to receive hook 53, and pocket 55 can be configured to receive hook 43. In some cases, hooks 43 and 53 can be placed within the walls of portions 41 and 51 so the hook ends come together when wrapped around catheter tubing. In some cases, hooks 43 and 53 can be placed within the walls of portions 41 and 51 so the hook ends are not near one another when wrapped around catheter tubing.

Device 40 can be composed of any appropriate material. For example, device 40 configured to hold tension and stabilize movement of a urethral catheter can be composed of material such that it has a rigid or semi-rigid structure. Examples of materials that can be used to make device 40 include, without limitation, polyvinyl chloride (PVC), silicone rubber, latex, Teflon, Nylon, and other appropriate material. Hooks 43 and 53 of device 40 can be composed of any appropriate material. For example, hooks 43 and 53 can be composed of material that is rigid. Examples of materials that can be used to make hooks 43 and 53 include, without limitation, metal wire, plastic, and other appropriate material. In some cases, hooks 43 and 53 can be coated to increase resistance to sliding of the catheter. Examples of materials that can be used to coat hooks 43 and 53 include, without limitation, polyvinyl chloride (PVC), silicone rubber, latex, adhesive, abrasives, and other appropriate material.

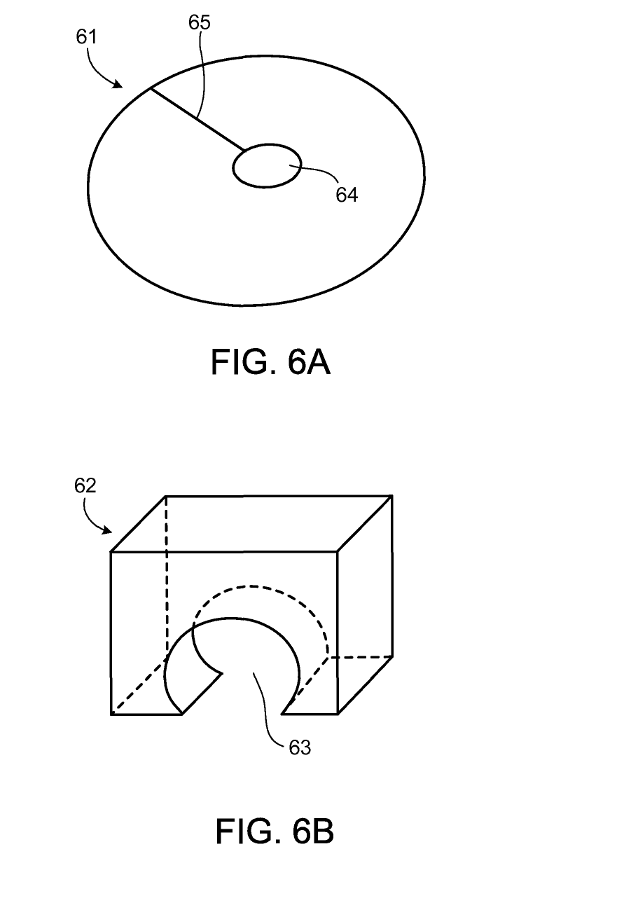

In some cases, a urethral catheter tension holding and movement stabilizing device can be composed of two individual portions (e.g., halves) that do not connect. For example, with reference to FIG. 6, a urethral catheter tension holding and movement stabilizing device can include a proximal component 61 that fits loosely on urethral catheter tubing and a distal component 62 that fits tightly on urethral catheter tubing. Distal component 62 can be of any appropriate shape with one portion thereof of side having a partial cylindrical channel 63 so as to firmly clamp around urethral catheter tubing and prevent the tubing from sliding. The diameter of channel 63 can vary to fit around different size urethral catheters. For example, the diameter can be between about 8 French and 30 French. Proximal component 61 can prevent contact between distal component 62 and the urethral meatus of the patient. Proximal component 61 can be any appropriate shape to provide patient comfort at the urethral meatus. For example, proximal component 61 can be a shape such as, without limitation, a disc, a semi-circular cup, a polygonal shape with or without rounded corners, or any other appropriate shape as to contain or rest against the penis glans, rest against the labia minora, labia majora, or any other perineal structure, or rest against the urethral meatus. In some cases, proximal component 61 can have perforations in its wall. Proximal component 61 can have a covering on a surface that contacts the patient for patient comfort. Proximal component 61 can have a lumen 64 through the center or another location of proximal component 61 for catheter tubing to fit through. Proximal component 61 can have a loose fit around catheter tubing. The diameter of lumen 64 can be between about 8 French to about 30 French. In some cases, proximal component 61 can have a slit 65 from a lumen to an outside edge so as to allow proximal component 61 to bend open to attach to catheter tubing.

A urethral catheter tension holding and movement stabilizing device provided herein that is composed of two individual components that do not connect can be composed of any appropriate material. For example, proximal component 61 can be composed of material such that it is flexible. Examples of materials that can be used to make proximal component 61 include, without limitation, polyvinyl chloride (PVC), silicone rubber, latex, Teflon, nylon, and other appropriate material. In some cases, distal component 62 provided herein can be composed of material such that it is rigid or semi-rigid. Examples of materials that can be used to make proximal component 61 include, without limitation, acrylic (plexiglass), polycarbonate, polyvinyl, flouroelastomers, acrylates, polystyrene, polyaryletheretherketone, polyphenylene sulphide, polypropylene, polyvinyl chloride (PVC), silicone, rubber, latex, Teflon, nylon, and other appropriate material. In some cases, a wall surface of partial cylindrical channel 63 of distal component 62 can be abrasive such that there is an increase in resistance to sliding of catheter tubing. In some cases, a wall surface of partial cylindrical channel 63 of distal component 62 can be lined with a material to increase resistance to sliding of catheter tubing such as, without limitation, a thin layer of silicone rubber or adhesive. In some cases, a wall surface of partial cylindrical channel 63 of distal component 62 can be made irregular to increase resistance to sliding of catheter tubing with configurations such as, without limitation, bumps, constriction bands, partial rings, teeth, or pins.

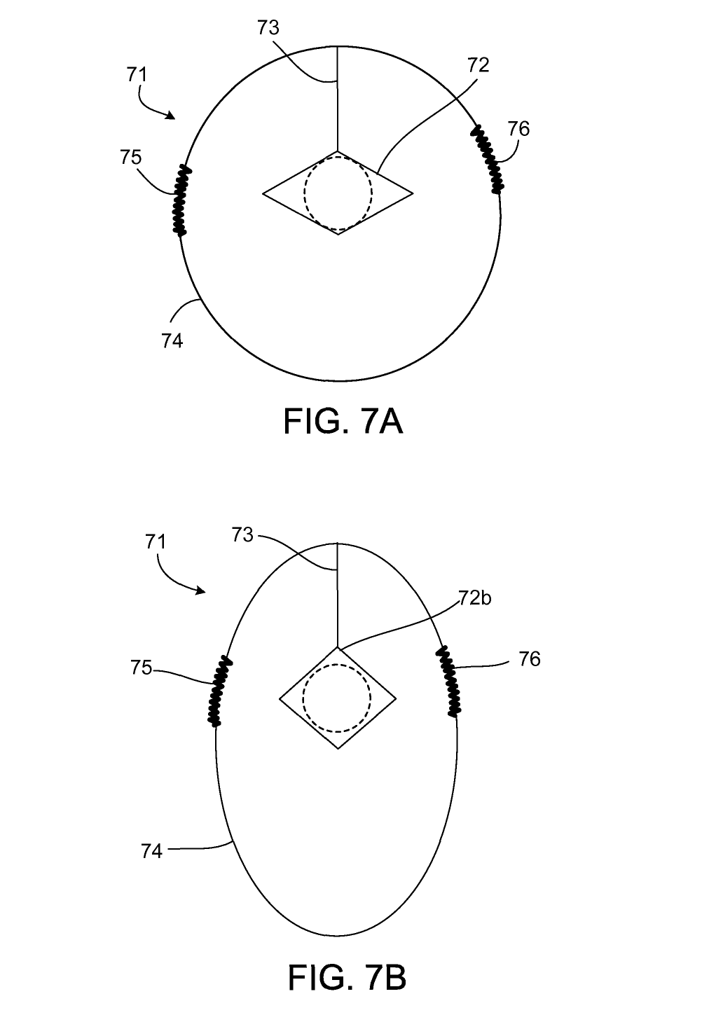

In some cases, a urethral catheter tension holding and movement stabilizing device provided herein can be composed of a single component. For example, with reference to FIG. 7, a urethral catheter tension holding and movement stabilizing device can consist of a component 71 with a lumen 72 configured to contain at least a portion of a urethral catheter tubing. Lumen 72 can be a shape such that the cross sectional length and width of lumen 72 are not equal and not all sides of lumen 72 grip catheter tubing. For example, lumen 72 can be a shape such as, without limitation, an oval, a parallelogram, or any other appropriate shape such that not all sides of lumen 72 contact catheter tubing. The longer cross sectional length of lumen 72 can be between about 8.1 French to about 60 French. The shorter cross sectional width of lumen 72 can be between about 7 French to about 32 French. Component 71 can contain a slit 73 extending from lumen 72 to an outer wall 74 so as to allow component 71 to bend open to attach to catheter tubing. Component 71 can slide along urethral catheter tubing towards the urethral meatus when force is applied on outer wall 74 and can lock in place on catheter tubing when force is released. Markings 75 and 76 located on outer wall 74 of component 71 can indicate the orientation of the longer length of lumen 72. When force is applied at markings 75 and 76, lumen 72 configures an open state 72b in which no walls (or a minimal portion of the walls) of lumen 72b contact urethral catheter tubing. Device 71 can move freely along catheter tubing when lumen 72b is in an open state. Lumen 72b can be configured to return to conformation 72 when force is released from markings 75 and 76 with at least two walls gripping catheter tubing, thus locking or securing component 71 in place.

Component 71 can be any appropriate shape to provide patient comfort at the urethral meatus. For example, component 71 can be a shape such as, without limitation, a disc, a semi-circular cup, a polygonal shape with or without rounded corners, or any other appropriate shape as to contain or rest against the penis glans, rest against the labia minora, labia majora, or any other perineal structure, or rest against the urethral meatus. Component 71 can have a diameter from about 1 cm to about 10 cm (e.g., from about 2 cm to about 10 cm, from about 3 cm to about 10 cm, from about 4 cm to about 10 cm, from about 1 cm to about 9 cm, from about 1 cm to about 8 cm, from about 1 cm to about 7 cm, or from about 2 cm to about 8 cm) and a thickness from about 1 mm to about 50 mm (e.g., from about 5 mm to about 50 mm, from about 10 mm to about 50 mm, from about 15 mm to about 50 mm, from about 1 mm to about 45 mm, from about 1 mm to about 40 mm, from about 1 mm to about 35 mm, or from about 10 mm to about 40 mm). If component 71 is a shape such that it can contain the penis glans, the length can range from flat to about 10 cm (e.g., from flat to about 9 cm, from flat to about 8 cm, from flat to about 7 cm, or from flat to about 6 cm). Component 71 can be composed of any appropriate material. For example, component 71 configured to hold tension and stabilize movement of a urethral catheter can be composed of material such that it has a semi-rigid structure. Examples of materials that can be used to make component 71 include, without limitation, polyvinyl chloride (PVC), silicone rubber, latex, Teflon, Nylon, and other appropriate material.

In some cases, a urethral catheter tension holding and movement stabilizing device provided herein can be an inflated balloon. For example, a balloon can be wrapped around a catheter tubing and attached to itself to form a ring surrounding the catheter. The balloon can be inflated to provide resistance to sliding of catheter tubing. The balloon can contain or rest against the penis glans, rest against the labia minora, labia majora, or any other perineal structure, or rest against the urethral meatus. In some cases, with reference to FIG. 6B, the balloon can function as distal component 62.

In some cases, a urethral catheter tension holding and movement stabilizing device can include a clip that can open and close and hold catheter tubing tightly when in a closed state. In some cases, an inner surface of such a clip can be lined with a material to increase resistance to sliding of catheter tubing such as, without limitation, a thin layer of silicone rubber, variations of surface texture, adhesive, or abrasives.

Any appropriate method can be used to hold tension on a urethral catheter using a device provided herein. For example, a physician can apply tension by pulling on a urethral catheter. In some cases, a urethral catheter tension holding and movement stabilizing device composed of two portions that connect to one another can be used to prevent a catheter tubing from slipping. For example, with reference to FIGS. 2, 3, and 4, portions 11 and 12 can be fastened together around a catheter tubing such that lumen 14 contains the catheter tubing and receptacle 15 holds the penis glans or rim 16 of receptacle 15 rests against the labia minora, labia majora, or any other perineal structure. In some cases, a urethral catheter tension holding and movement stabilizing device that includes two individual portions (e.g., halves) that can grip a catheter tubing can be used to prevent a catheter tubing from slipping. With reference to FIG. 5, portions 41 and 51 can be placed on opposite sides of a catheter tubing, with hooks 43 and 53 placed around the catheter tubing. Portions 41 and 51 can be moved adjacent to one another with the catheter tubing contained between them in lumen 42. Device 40 can be slid along catheter tubing when force is applied to markings 44 and 54, releasing the grip of hooks 43 and 53 on the catheter tubing. When force is released from markings 44 and 54, hooks 43 and 53 move back into position to grip the catheter tubing. In some cases, device 40 can be applied to the catheter tubing, and the two portions 41 and 51 can be squeezed to slide device 40 into position external to the urethral meatus and released to prevent the catheter tubing from slipping.

In some cases, a urethral catheter tension holding and movement stabilizing device that includes two individual portions (e.g., halves) that do not connect can be used to prevent the catheter tubing from slipping. With reference to FIG. 6, proximal piece 61 can be bent at slit 65 to create an opening from an outside edge to lumen 64. In some cases, proximal piece 61 can fit onto catheter tubing through this opening such that the catheter tubing passes through lumen 64. Proximal piece 61 can be made of a flexible material for patient comfort as this piece is external to the urethral meatus. Proximal piece 61 can move easily along catheter tubing. Once proximal piece 61 is in place, distal piece 62 can be clamped onto the catheter tubing just distal to proximal piece 61. Distal piece 62 can prevent a catheter tubing from slipping, while proximal piece 63 can provide patient comfort.

In some cases, a urethral catheter tension holding and movement stabilizing device that is configured as a single component can be used to prevent a catheter tubing from slipping. With reference to FIG. 7, component 71 can be bent at slit 73 to create an opening from outer wall 74 to lumen 72. Component 71 can slide on a urethral catheter tubing towards the urethral meatus when force is applied on outer wall 74 at markings 75 and 76 and can hold tightly onto a catheter tubing when force is released.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A device for holding tension on or stabilizing the movement of a urethral catheter, wherein said device comprises two separable components configured to be attached to a urethral catheter tubing, thereby forming said device, wherein said device is configured to contain or rest against the penis glans or rest against the labia minora, labia majora, or other perineal structure, wherein at least one of said two separable components comprises a fastener configured to hold said two separable components together, and wherein formation of said device forms a continuous open tube configured to retain at least a portion of said urethral catheter tubing.

2. The device of claim 1, wherein an inner surface of said continuous open tube configured to retain at least a portion of said urethral catheter tubing is coated.

3. The device of claim 1, wherein an inner surface of said continuous open tube configured to retain at least a portion of said urethral catheter tubing is coated with a material selected from the group consisting of polyvinyl chloride (PVC), silicone, rubber, latex, Teflon, nylon, adhesives, and abrasives.

4. The device of claim 1, wherein an inner surface of said continuous open tube configured to retain at least a portion of said urethral catheter tubing is not smooth.

5. The device of claim 1, wherein an inner surface of said continuous open tube configured to retain at least a portion of said urethral catheter tubing comprises a ring, a bump, teeth, a hook, or a pin.

6. The device of claim 1, wherein said fastener comprises a complimentary male protrusion and female indentation.

7. The device of claim 1, wherein said fastener comprises at least two magnets.

8. The device of claim 1, wherein said device comprises material selected from the group consisting of polyvinyl chloride (PVC), silicone, rubber, latex, Teflon, and Nylon.

9. The device of claim 1, wherein a diameter of said continuous open tube configured to retain at least a portion of said urethral catheter tubing is between 8 French and 30 French.

10. The device of claim 1, wherein said diameter is between 18 French and 24 French.

11. (canceled)

12. A device for holding tension on or stabilizing the movement of a urethral catheter, wherein said device comprises two separable components configured to be attached to a urethral catheter tubing, thereby forming said device, wherein said device is configured to sit external to the urethral meatus of a human patient, wherein formation of said device forms a lumen configured to retain at least a portion of said urethral catheter tubing, wherein said two separable components are configured to contain at least one hook configured to grip said catheter tubing, wherein force applied to the external surface of said device moves said hook to release said hook from said catheter tubing, wherein said device displays at least two markings on said external surface, and wherein said markings indicate where said force is to be applied.

13. The device of claim 12, wherein an inner surface of said lumen is coated.

14. The device of claim 1, wherein an inner surface of said lumen is coated with a material selected from the group consisting of polyvinyl chloride (PVC), silicone, rubber, latex, Teflon, Nylon, adhesives, and abrasives.

15. The device of claim 1, wherein said hook comprises metal wire or plastic.

16. The device of claim 1, wherein said device comprises material selected from the group consisting of polyvinyl chloride (PVC), silicone, rubber, latex, Teflon, and Nylon.

17. The device of claim 1, wherein a diameter of said lumen is between 8 French and 30 French.

18. The device of claim 1, wherein a diameter of said lumen is between 18 French and 24 French.

19. (canceled)

20. A device for holding tension on or stabilizing the movement of a urethral catheter, wherein said device comprises a flexible proximal component and a rigid distal component configured to be attached to a urethral catheter tubing, thereby forming said device, wherein said device is configured to sit external to the urethral meatus of a human patient, wherein said distal component is configured to retain at least a portion of said urethral catheter tubing, and wherein said proximal component separates said distal component and said urethral meatus of said patient.

21. The device of claim 20, wherein said proximal component comprises material selected from the group consisting of polyvinyl chloride (PVC), silicone, rubber, latex, Teflon, and Nylon.

22. The device of claim 1, wherein said distal component comprises material selected from the group consisting of acrylic (plexiglass), polycarbonate, polyvinyl, flouroelastomers, acrylates, polystyrene, polyaryletheretherketone, polyphenylene sulphide, polypropylene, polyvinyl chloride (PVC), silicone, rubber, latex, Teflon, and Nylon.

23-62. (canceled)
